(19) United States
(12) Patent Application Publication
(10) Pub. No.: US 2025/0352768 A1
(43) Pub. Date: Nov. 20, 2025

(54) GUIDE CATHETER FOR AN IMPLANT DELIVERY APPARATUS

(71) Applicant: EDWARDS LIFESCIENCES CORPORATION, Irvine, CA (US)

(72) Inventors: Darshin S. Patel, Lake Forest, CA (US); Alana Tyler Stein, Irvine, CA (US)

(21) Appl. No.: 19/284,409

(22) Filed: Jul. 29, 2025

Related U.S. Application Data
(63) Continuation of application No. PCT/US2024/013282, filed on Jan. 29, 2024.
(60) Provisional application No. 63/482,204, filed on Jan. 30, 2023.

(51) Int. Cl. A61M 25/06; A61F 2/24; A61M 25/00; A61M 25/01

(52) U.S. Cl. CPC A61M 25/0662; A61F 2/2427; A61M 25/0043; A61M 25/007; A61M 25/0136; A61M 2025/0681; A61M 2210/125

(57) ABSTRACT

Devices and methods for equalizing pressure in a main lumen of a guide sheath shaft are disclosed. As one example, a delivery apparatus can include a handle and a shaft extending within and distally from the handle. The shaft has a main lumen, and a distal end portion of the shaft includes one or more holes that extend through a wall of the shaft, between the main lumen and an exterior of the shaft. The one or more holes are spaced axially away from a distal end of the shaft.

### CROSS-REFERENCE TO RELATED APPLICATION

This application is a continuation of PCT Patent Application No. PCT/US2024/013282 filed on Jan. 29, 2024, which claims the benefit of U.S. Provisional Patent Application No. 63/482,204, filed Jan. 30, 2023, each of these applications being incorporated by reference herein in its entirety.

### FIELD

The present disclosure relates to guide catheters for delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

A guide catheter (which can also be referred to as a guide sheath) can be used for introducing an implant delivery apparatus, such as the prosthetic heart valve delivery apparatus described above, into the patient's vasculature. The guide catheter can include an elongated shaft that is inserted into the vasculature and a handle that remains outside the patient and can be used to manipulate the shaft. The implant delivery apparatus can be inserted through a lumen of the guide catheter to help direct the implant delivery apparatus to a target implantation site (e.g., a native valve region) within the patient and/or help position the implant delivery apparatus at the target implantation site.

### SUMMARY

Described herein are prosthetic heart valves, docking devices, delivery apparatuses, guide catheters, and methods for implanting docking devices and prosthetic heart valves. The disclosed guide catheters can, for example, be configured to receive a portion of a delivery apparatus within a main lumen of the guide catheter in order to introduce the delivery apparatus into a patient's vasculature and guide the delivery apparatus toward a target implantation site for a prosthetic medical device mounted on the delivery apparatus. In some examples, the guide catheter can include one or more through-holes or channels that extend between the main lumen and an exterior of the guide catheter, the one or more through-holes disposed in a distal end portion of the guide catheter. As such, the devices and methods disclosed herein can, among other things, overcome one or more of the deficiencies of typical guide catheters.

A delivery apparatus can comprise a handle and one or more shafts coupled to the handle.

In some examples, a delivery apparatus can comprise a handle and a shaft extending distally from the handle, the shaft comprising a main lumen and one or more holes disposed in a distal end portion of the shaft.

In some examples, the one or more holes extend through a wall of the shaft, between the main lumen and an outer surface of the shaft.

In some examples, the one or more holes is spaced axially away from a distal end of the shaft.

In some examples, the one or more holes includes a plurality of spaced apart holes.

In some examples, the plurality of spaced apart holes is arranged in a helical pattern around the shaft, along a portion of a length of the shaft.

In some examples, a delivery apparatus comprises a handle, and a shaft extending distally from the handle and having a main lumen. A distal end portion of the shaft comprises one or more holes that extend through a wall of the shaft, between the main lumen and an exterior of the shaft, and the one or more holes are spaced axially away from a distal end of the shaft.

In some examples, a delivery apparatus comprises one or more of the components recited in Examples 1-13 and 33-35 below.

A delivery assembly can comprise an implant catheter and a guide catheter.

In some examples, the guide catheter can comprise a handle and a shaft extending distally from the handle, the shaft having a main lumen that is configured to receive a portion of the implant catheter therethrough.

In some examples, a distal end portion of the shaft can comprise one or more holes extending through the shaft, between the main lumen and an outer surface of the shaft.

In some examples, the one or more holes are spaced away from a distal end of the shaft.

In some examples, the one or more holes include a plurality of spaced apart holes.

In some examples, the plurality of spaced apart holes is arranged in a helical pattern around the shaft, along a portion of a length of the shaft.

In some examples, a delivery assembly comprises an implant catheter, and a guide catheter. The guide catheter comprises a handle, and a shaft extending distally from within the handle and having a main lumen that is configured to receive a portion of the implant catheter therethrough. A distal end portion of the shaft includes one or more through-holes therein that each extend through a wall of the shaft, between the main lumen and an outer surface of the shaft, and the one or more through-holes are spaced axially away from a distal end of the shaft.

In some examples, a delivery assembly comprises one or more of the components recited in Examples 14-22 and 36-37 below.

A guide sheath can comprise a handle and a shaft extending distally from the handle.

In some examples, the shaft can comprise a main lumen defined by an inner surface of a wall of the shaft.

In some examples, a distal end portion of the shaft can comprise one or more holes extending through the wall of the shaft, between the inner surface and an outer surface of the wall of the shaft.

In some examples, the one or more holes includes a plurality of spaced apart holes.

In some examples, the plurality of spaced apart holes is arranged in a helical pattern around the shaft, along a portion of a length of the shaft.

In some examples, the one or more holes are spaced axially away from a distal end of the shaft.

In some examples, a guide sheath comprises a handle comprising a seal housing assembly including one or more fluid seals, and a shaft extending within and distally from the handle and having a main lumen that extends within the housing and through the seal housing assembly. A distal end portion of the shaft includes a plurality of holes that extend through a thickness of a wall of the shaft, between the main lumen and an outer surface of the shaft, and the plurality of holes is spaced axially away from a distal end of the shaft.

In some examples, a guide sheath comprises one or more of the components recited in Examples 23-29 and 38-39 below.

In some examples, a method comprises inserting a shaft of a guide catheter into a vessel of a patient and advancing a distal end portion of a shaft of the guide shaft into the heart of the patient such that a distal end of the shaft is positioned in the left atrium of the heart and one or more through-holes in the distal end portion of the shaft are positioned in the right atrium of the heart, inserting a distal end portion of a first implant catheter into a proximal end of the guide catheter and pushing the distal end portion of the first implant catheter through a main lumen of the guide catheter toward a target implantation site for a prosthetic medical device mounted on the distal end portion of the first implant catheter, and as the prosthetic medical device mounted on the distal end portion of the first implant catheter passes by the one or more through-holes in the shaft, releasing fluid traveling behind the prosthetic medical device into the right atrium through the one or more through-holes.

In some examples, the fluid is air.

In some examples, the method comprises one or more of the features recited in Examples 30-32 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a perspective view of an exemplary delivery apparatus for a prosthetic heart valve.

FIG. 6 is side view of an exemplary guide catheter configured to receive a delivery apparatus and guide the delivery apparatus through a portion of a patient's vasculature.

FIG. 7 is a cross-sectional side view of a proximal end portion of the guide catheter of FIG. 6.

FIG. 8 is a cross-sectional side view of a distal end portion of the guide catheter of FIG. 6.

FIG. 9 is a schematic illustrating the guide catheter of FIG. 6 positioned in the heart such that apertures in the distal end portion of the guide catheter shaft are disposed in the right atrium of the heart.

FIG. 10 is a side view of a distal end portion for a guide catheter including holes of different sizes spaced apart therein.

FIG. 11 is a side view of a distal end portion for a guide catheter including a plurality of spaced apart holes in a helical arrangement along the distal end portion.

FIG. 12 is a side view of a distal end portion for a guide catheter including a plurality of spaced part holes in a helical arrangement along the distal end portion.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, a guide catheter can be inserted into a patient's vasculature and then receive an implant delivery apparatus within a main lumen of the guide catheter in order to direct the delivery apparatus therethrough to a target implantation site for a prosthetic implant. In some examples, an inner diameter of the main lumen of the guide catheter and an outer diameter of portions of the implant delivery apparatus can be closely matched. Thus, in some examples, as the delivery apparatus is pushed distally through the main lumen of the guide catheter, a negative pressure (or vacuum) can be created within the main lumen, proximal to the implant, thereby creating an increase in a pressure gradient across one or more fluid seals within a handle of the guide catheter. This can also result in an increase in a force felt by a user as they push the delivery apparatus through the guide catheter (referred to as “push forces”). Accordingly, improvements to the guide catheter that decrease or prevent negative pressure from being created within the main lumen are desirable. Such improvements can, for example, help maintain hemostasis and/or reduce push forces when advancing a delivery apparatus through the guide catheter.

Described herein are various systems, apparatuses, methods, or the like, that, in some examples, can be used in or with delivery apparatuses for prosthetic medical devices (such as prosthetic heart valves or docking devices). In some examples, such systems, apparatuses, and/or methods can provide a shaft of a guide catheter with one or more through-holes or channels that are configured to equalize pressure within a main lumen of the shaft as a delivery apparatus is navigated through the main lumen of the guide catheter toward an implantation site in a body of a patient. The through-holes in the shaft can equalize negative pressure created within the system, thereby reducing push forces felt by a user pushing the delivery apparatus through the guide catheter. In some examples, in the event that there is residual air in the system, the residual air can be released via the through-holes along a specified portion of the guide catheter shaft (e.g., in the right heart such that the air is expelled to the lungs). As a result, the system can be easier to operate.

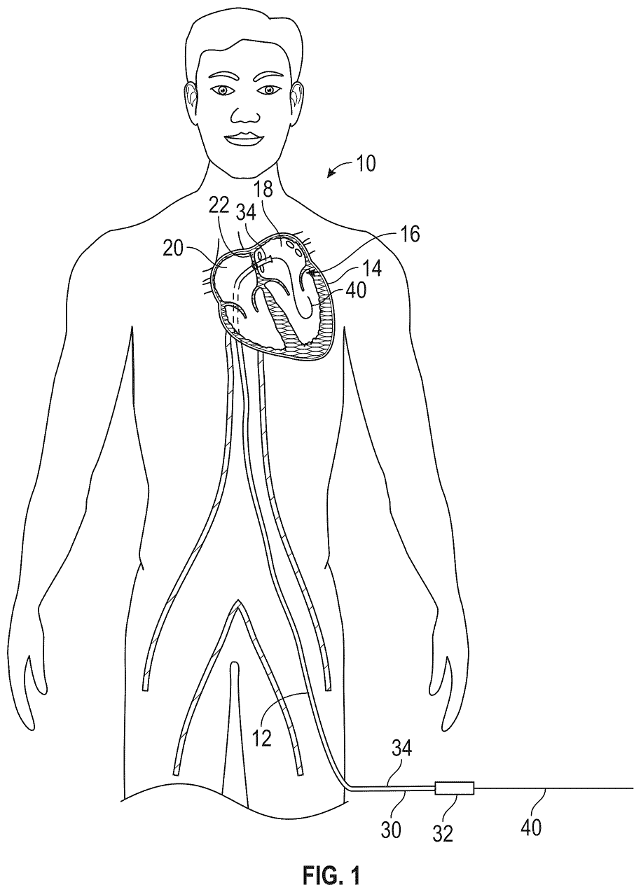

In some examples, the guide catheters disclosed herein can be used to introduce one or more delivery apparatuses (or implant catheters) into the vasculature of a patient and guide the one or more delivery apparatuses at least partially through the vasculature toward a target implantation site. For example, FIGS. 1-4 schematically illustrate an exemplary transcatheter heart valve replacement procedure which utilizes a guide catheter to guide a docking device delivery apparatus toward a native valve annulus and then a prosthetic heart valve delivery apparatus toward the native valve annulus. The docking device delivery apparatus is used to deliver a docking device to the native valve annulus. The prosthetic heart valve delivery apparatus is used to deliver a transcatheter prosthetic heart valve inside the docking device.

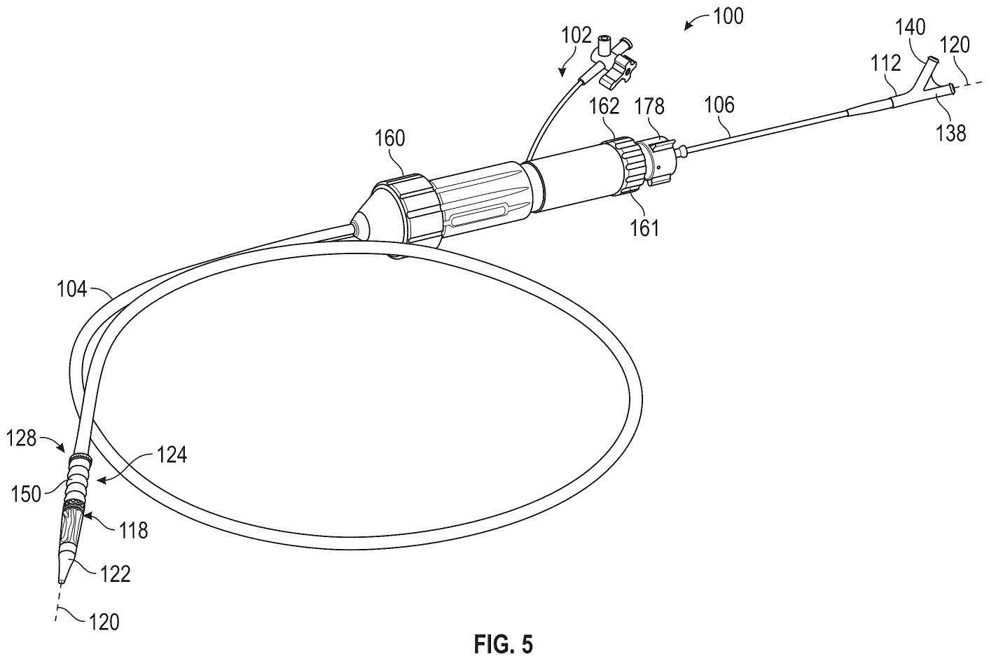

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve. An exemplary delivery apparatus for delivery a prosthetic heart valve within a docking device at a native heart valve is shown in FIG. 5.

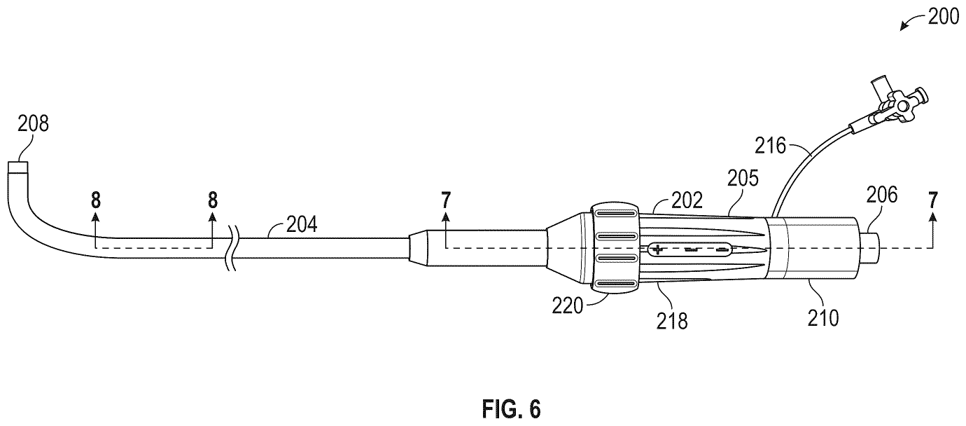

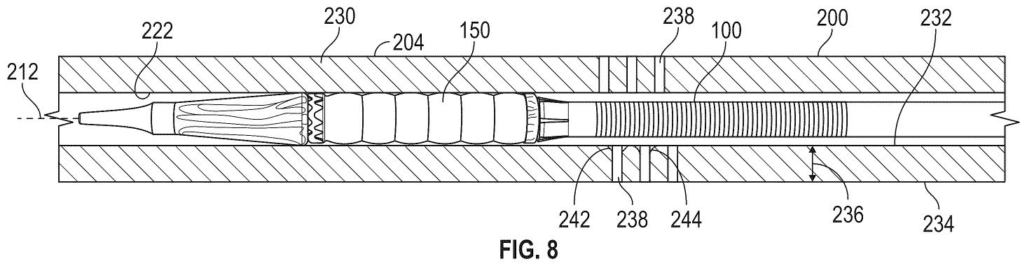

An exemplary guide catheter is shown in more details in FIGS. 6-8. In some examples, as shown in FIG. 8, the guide catheter can include one or more channels or through-holes extending through the guide catheter shaft, between a lumen and exterior of the shaft. As such, vacuum pressure created by advancing a prosthetic implant on a delivery apparatus through the lumen of the guide catheter can be equalized and, if any air is present within the shaft, it can be released at a specified location along the shaft. As a result, push forces felt by a user operating the delivery apparatus can be reduced and, in some instances, any air present within the system can be released in an effective manner.

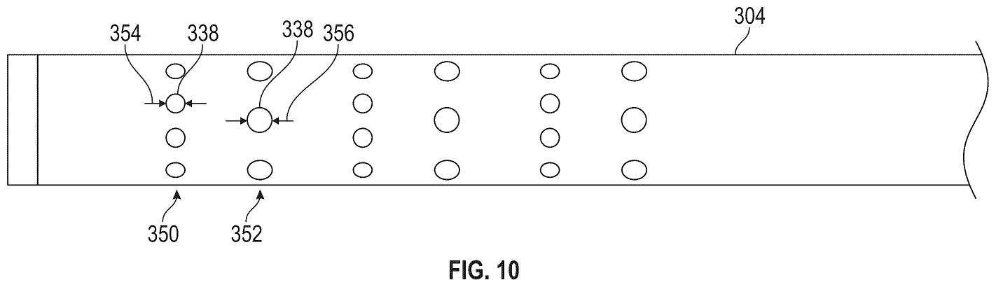

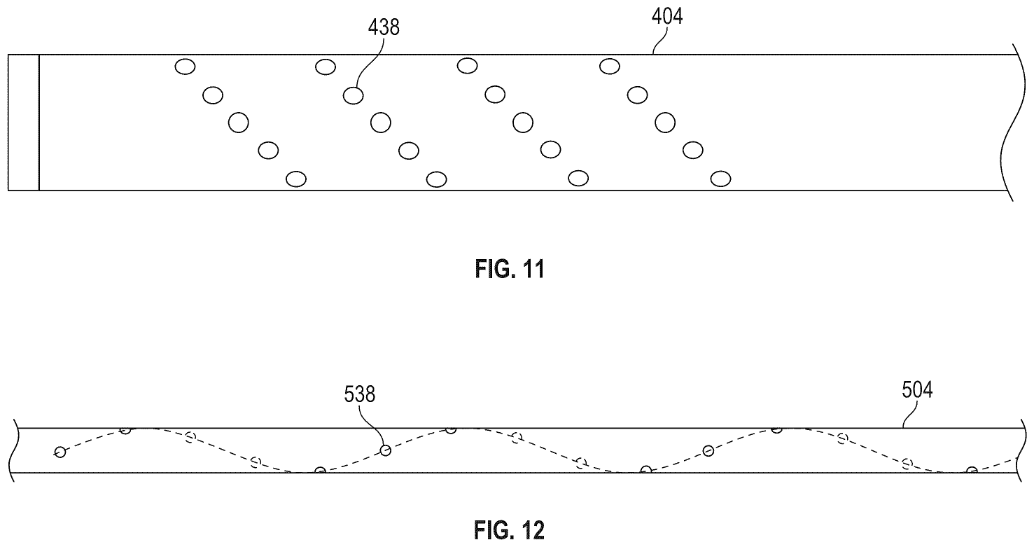

In some examples, as shown in FIG. 10, the through-holes can have different sizes (for example, diameters). In some examples, the through-holes can be arranged in a pattern of annular rings of spaced-apart holes (as shown in FIG. 10) or in a helical arrangement of spaced-apart holes (as shown in FIGS. 11 and 12). As a result, no matter the rotational orientation of the guide catheter shaft, at least a portion of the holes will be oriented such that, if there is any air present, it can be released through the portion of the holes.

### Examples of the Disclosed Technology

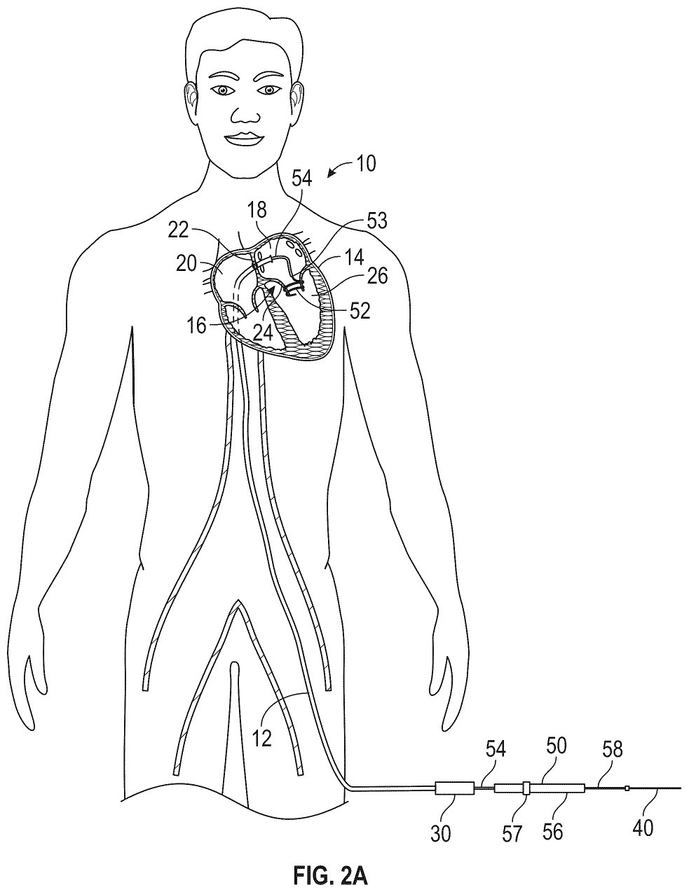

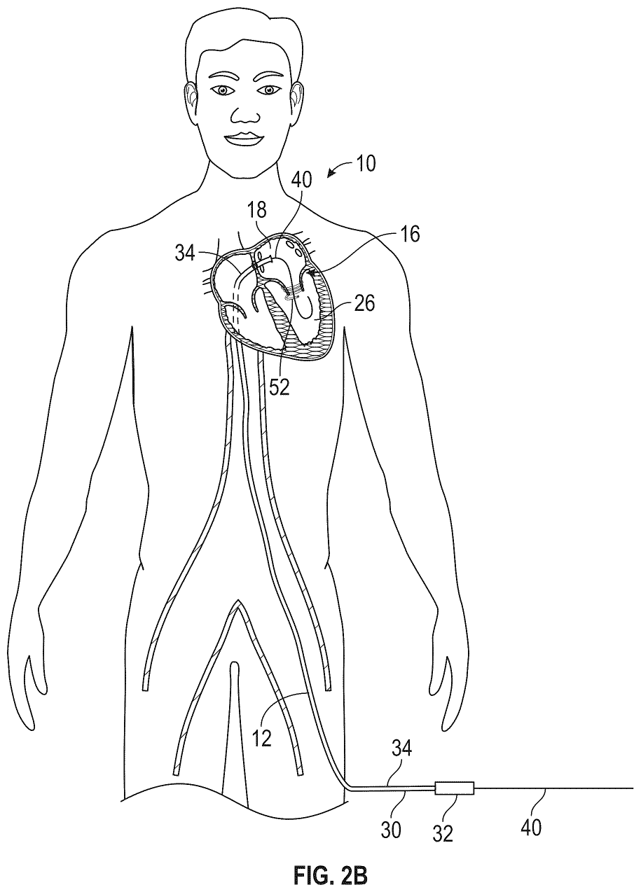

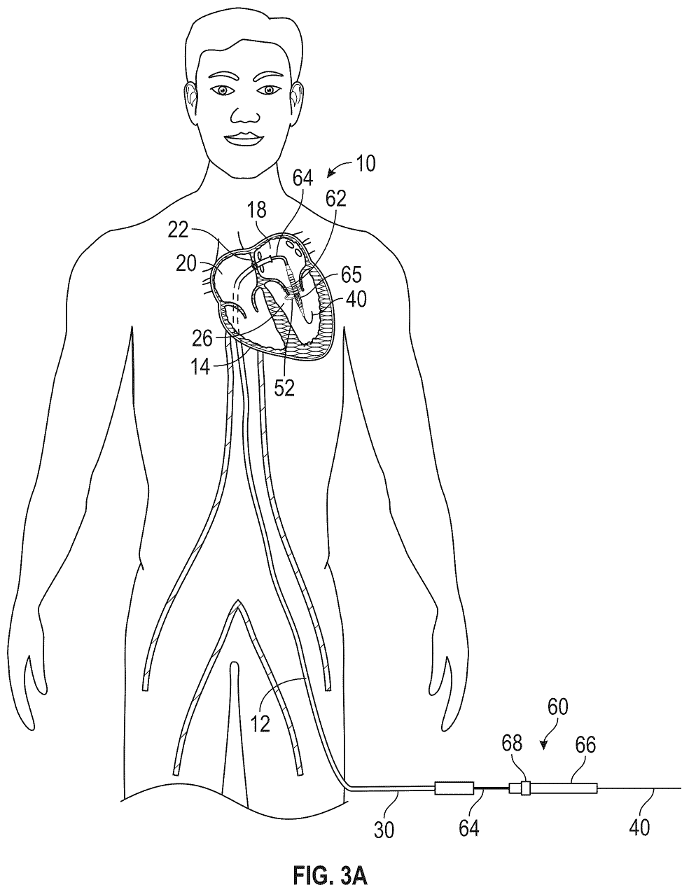

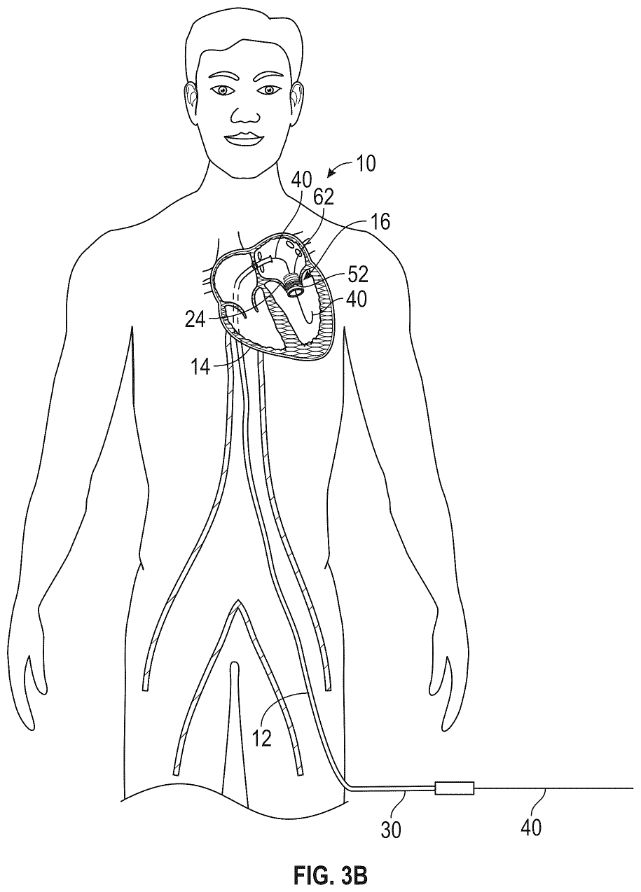

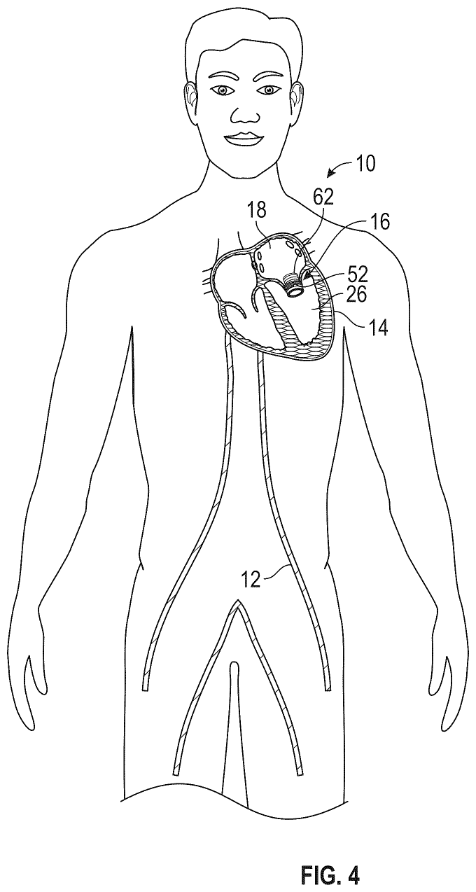

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device”, “introducer”, or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can make a small incision in an atrial septum 22 of the heart 14 to allow access to the left atrium 18 from the right atrium 20. The user can insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In some examples, the prosthetic heart valve 62 can be self-expanding and can be configured to radially expand on its own upon removable of a sheath or capsule covering the radially compressed prosthetic heart valve 62 on the distal end portion of the delivery shaft 64.

In some examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and remove the delivery shaft 54 of the docking device delivery apparatus 50 from the patient 10. The user may advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may expand the prosthetic heart valve 62 within the docking device 52 before removing the prosthetic valve delivery apparatus 60 from the patient 10. In some examples, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and through the aortic valve into the left ventricle 26.

FIG. 5 illustrates an exemplary prosthetic heart valve delivery apparatus 100 (which can also be referred to here as an “implant catheter”) that can be used in lieu of the prosthetic valve delivery apparatus 60 of FIG. 3A to implant an expandable prosthetic heart valve. In some examples, the delivery apparatus 100 is specifically adapted for use in introducing a prosthetic heart valve into a heart.

The delivery apparatus 100 in the illustrated example of FIG. 5 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. In some examples, the delivery apparatus 100 can further comprise an inner shaft extending distally from the handle 102 coaxially through the intermediate shaft 106 and the outer shaft 104 and proximally from the handle 102 coaxially through the intermediate shaft.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion that extends proximally from a proximal end of the handle 102, to an adaptor 112. The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

In some examples, the intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft can extend distally beyond the distal end portion of the intermediate shaft 106 toward or to a nose cone 122 at a distal end of the delivery apparatus 100.

In some examples, a distal end of the balloon 118 can be coupled to a distal end of the delivery apparatus 100, such as to the nose cone 122 (as shown in FIG. 5), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 (shown in FIG. 5) can overly a distal shoulder of the delivery apparatus 100. As shown in FIG. 5, a prosthetic heart valve 150 can be mounted around the balloon 118, at the valve mounting portion 124 of the delivery apparatus 100, in a radially compressed state. The prosthetic heart valve 150 can be configured to be radially expanded by inflation of the balloon 118 at a native valve annulus, as described above with reference to FIGS. 3A and 3B.

A balloon shoulder assembly of the delivery apparatus 100, which includes the distal shoulder, is configured to maintain the prosthetic heart valve 150 (or other medical device) at a fixed position on the balloon 118 during delivery through the patient's vasculature.

The outer shaft 104 can include a distal tip portion 128 (best seen in FIG. 8) mounted on its distal end. In some examples, the outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 5) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site).

Turning now to FIGS. 6-8, an exemplary guide catheter, which is referred to below as a guide sheath 200 (and can also be referred to herein as a “delivery apparatus” or an “introducer device” or an “introducer”) is shown. In some examples, the guide sheath 200 can be used in lieu of the guide catheter 30 in a docking device and/or a prosthetic valve implantation procedure, as described above with reference to FIGS. 1-4. The guide sheath 200 can be configured to be inserted into a patient's vasculature and receive an implant catheter or delivery apparatus therein (e.g., such as the delivery apparatus 100 of FIG. 5, as shown in FIG. 8) in order to introduce the implant catheter into the patient's vasculature and at least partially guide the implant catheter therein to a target implantations site. Though the guide sheath 200 is described herein as being used with the delivery apparatus 100, the guide sheath 200 can be configured to receive a variety of delivery apparatuses or implant catheters, such as alternate prosthetic heart valve delivery apparatuses, docking device delivery apparatuses, and/or delivery apparatuses for other prosthetic medical devices or medical therapies, such as stents.

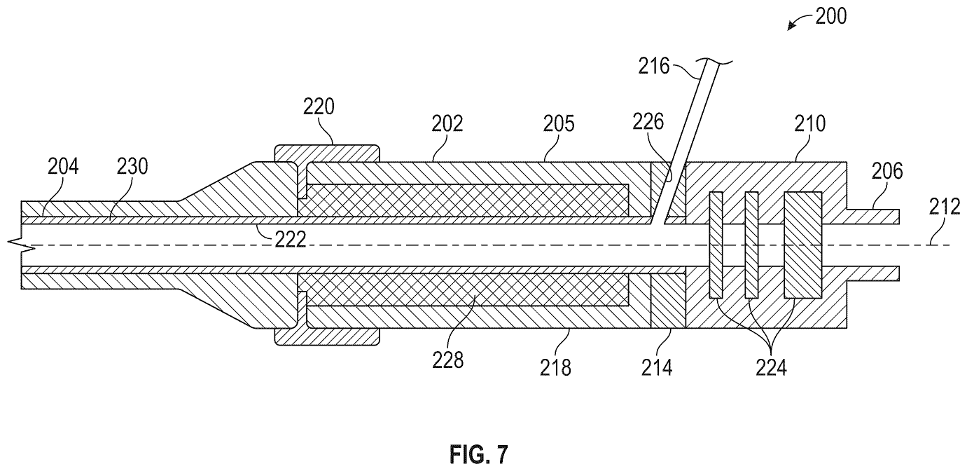

The guide sheath 200 in the illustrated example comprises a handle 202, an elongated shaft 204 extending distally from the handle 202 (FIGS. 6 and 7), and a central longitudinal axis 212 (FIG. 7). The shaft 204 has a main (or primary) lumen 222 that is defined by an inner surface of a wall 230 of the shaft 204 (FIGS. 7 and 8). The main lumen 222 is configured to receive a delivery apparatus therein (such as any of the prosthetic device delivery apparatuses or implant catheters described herein). In some examples, as shown in FIG. 7, the shaft 204 can extend into the handle 202. Further, in some examples, the main lumen 222 can extend through the handle 202 to an inlet port 206 disposed at a proximal end of the handle 202. Thus, in some examples, an inner surface of a wall of a portion of the handle (e.g., at the proximal end) can further define the main lumen 222. Thus, in some instances, the main lumen 222 can extend from the inlet port 206 to a distal end 208 of the shaft 204 (FIG. 6).

The handle 202 can have a housing 205 (also referred to as an “outer housing 205”) comprising a main body portion 218 and a seal housing assembly 210 (which can also be referred to as a “seal stack”) which comprises one or more seals 224 contained therein (FIGS. 6 and 7). The one or more seals 224 of the seal housing assembly 210 can be configured to fluidly seal the main lumen 222 of the guide sheath 200 from the external environment. For example, the one or more seals 224 of the seal housing assembly 210 can be configured to prevent blood from a patient in which the guide sheath 200 is inserted from exiting the guide sheath 200 and prevent air from the environment from entering the guide sheath 200 (e.g., through the inlet port 206). The one or more seals 224 can include a variety of types of seals, such as a duckbill seal, a flapper seal, an umbrella valve, a cross-slit valve, a dome valve, or the like.

The main body portion 218 is disposed adjacent and distal to the seal housing assembly 210. The handle 102 can, in some instances, include an adaptor spine 214 disposed adjacent and distal to the seal housing assembly 110 (FIG. 7). In this way, the adaptor spine 214 can either form a proximal portion of the main body portion 218 or be disposed between the main body portion 218 and the seal housing assembly 210.

A flush port 216 can be connected to the housing 205 at the adaptor spine 214. A flush lumen 226 of the adaptor spine 214 is connected to the flush port 216 and further connects to the main lumen 222 (FIG. 7). The flush port 216 can be configured to receive fluid through a lumen thereof. In this way, the flush port 216 can be fluidly coupled to the main lumen 222 by the flush lumen 226.

The handle 202 can include a steering mechanism configured to adjust the curvature of the distal end portion of the shaft 204 (as such, the shaft 204 can be referred to as a steerable shaft). In the illustrated example, the handle 202 includes an adjustment member, such as the illustrated rotatable knob 220 (FIGS. 6 and 7). The main body portion 218 can house internal flex mechanisms 228 of the guide sheath 200 which are operatively coupled to the rotatable knob 220 (FIG. 7). In some examples, the flex mechanisms 228, and thus the knob 220, can be operatively coupled to the proximal end portion of a pull wire. The pull wire can extend distally from the handle 202 through the shaft 204 and have a distal end portion affixed to the shaft 204 at or near the distal end 208 of the shaft 204. Rotating the knob 220 can increase or decrease the tension in the pull wire, thereby adjusting the curvature of the distal end portion of the shaft 204. Further details on steering or flex mechanisms for a delivery apparatus can be found in U.S. Pat. No. 9,339,384, as already incorporated by reference above.

FIG. 8 is a cross-sectional side view of the shaft 204 of the guide sheath 200, taken along the section line 8-8 shown in FIG. 6. Thus, FIG. 8 shows a side cross-section of a distal end portion of the shaft 204 (e.g., a portion of the shaft 204 that is disposed closer to the distal end 208 than to the handle 202).

As introduced above, the main lumen 222 of the shaft 204 is defined by an inner surface 232 of the wall 230 of the shaft 204. In some instances, the shaft 204 is annular and the wall 230 has the inner surface 232 (e.g., a radially inward facing surface relative to the central longitudinal axis 212) and outer surface 234 (e.g., a radially outward facing surface) with a thickness 236 of the wall 230 defined therebetween.

As shown in FIG. 8, the distal end portion of the shaft 204 can include one or more holes 238 (which can also be referred to herein as through-holes, channels, and/or apertures) which extend through the wall 230, from the main lumen 222 to an exterior of the shaft 204. For example, each hole 238 can extend through the thickness 236 of the wall 230, between the inner surface 232 and the outer surface 234. In this way, a length of each hole 238 can be at least as great as the thickness 236 of the wall 230.

In some examples, the one or more holes 238 are radially extending holes.

In some examples, the distal end portion of the shaft 204 can comprise a plurality of holes 238.

In some instances, at least a portion of the multiple holes 238 can be spaced axially apart from one another along the distal end portion of the shaft 204.

In some instances, at least a portion of the multiple holes 238 can be spaced circumferentially apart from one another around the shaft 204.

In some examples, each hole 238 can be spaced circumferentially apart from at least one other hole 238 and axially apart from at least one other hole 238.

In some examples, the holes 238 can be disposed in the shaft 204 at various circumferential locations around the shaft 204 such that in any rotational position of the shaft 204, when inside a patient's vessel, at least one hole 238 of multiple holes 238 is facing upward (relative to the ground). As a result, fluid (such as air) traveling through the shaft 204 can exit more easily through one or more holes 238 (since air tends to travel upward, or to a highest point).

In some instances, a circumferential placement of the holes 238 in the shaft 204 can cover a full 360 degrees around the shaft 204 along a portion of a length of the shaft 204.

For example, in some instances, a subset of the holes 238 can be axially aligned and circumferentially spaced around the shaft 204 such that they form a ring of spaced apart holes 238 around a specified axial location of the shaft 204. One or more such rings of holes 238 can be formed along the shaft 204.

For example, as shown in FIG. 10, a distal end portion of a shaft 304 for a guide sheath (such as guide sheath 200) can include multiple rings 350, 352 of circumferentially spaced apart holes 338. The holes 338 can be the same or similar to the holes 238, as described above. Further, in some examples, the shaft 304 can be used in lieu of the shaft 204 in guide sheath 200. Each ring 350 and/or 352 is spaced axially apart from at least one adjacent ring 350 and/or 352, along a length of the distal end portion of the shaft 304.

In some examples, at least one hole 338 can have a different diameter or width than another hole 338 in the distal end portion of the shaft 304 (as shown in FIG. 10). For example, a first portion of holes 338 can have a first diameter 354 and a second portion of holes 338 can have a second diameter 356.

As shown in the example of FIG. 10, in some examples, a first ring 350 of holes 338 can includes holes 338 having the first diameter 354 and a second ring 352 of holes 338 can include holes 338 having the second diameter 356, wherein the second diameter 356 is larger than the first diameter 354.

In some examples, at least one ring 350, 352 can have holes 338 of varying diameters or width.

In some examples, the rings 350, 352 can have varying numbers of holes 338. For example, as shown in example of FIG. 10, the first ring 350 can have a greater number of holes 338 than the second ring 352.

In some instances, a subset (or all) of the holes 238 can form a helix around the shaft 204, along a portion of a length of the shaft 204. The helix of holes 238 can form one or more revolutions around the shaft 204 (e.g., at least one revolution, two revolutions, three revolutions, or the like). In some examples, the helix of holes 238 can form two or more revolutions around the shaft 204.

For example, FIG. 11 shows a schematic side view of a distal end portion of a shaft 404 for a guide sheath (such as guide sheath 200) with a helical arrangement (or pattern) of holes 438 around the shaft 404, along a length of at least a portion of the distal end portion of the shaft 404 (holes 438 “behind” the shaft 404 are not visible in FIG. 11). The holes 438 can be the same or similar to the holes 238, as described above. Further, in some examples, the shaft 404 can be used in lieu of the shaft 204 in guide sheath 200.

The holes 438 arranged in the helical pattern can have various spacings from one another and/or various revolutions around the shaft 404. In the example shown in FIG. 4, the holes 438 form at least three revolutions around the shaft 404 and a pitch of the helix is relatively small or tight.

FIG. 12 depicts a schematic view of a distal end portion of a shaft 504 for a guide sheath (such as guide sheath 200) with a helical arrangement (or pattern) of holes 538 around the shaft 504, along a length of at least a portion of the distal end portion of the shaft 504 (the holes 538 “behind” the shaft 504 are depicted with dashed lines in FIG. 12). The holes 538 can be the same or similar to the holes 238, as described above. Further, in some examples, the shaft 504 can be used in lieu of the shaft 204 in guide sheath 200.

In the example of FIG. 12, the helix of holes 538 completes at least two revolutions around the shaft 504. As shown in FIG. 12, the pitch of the helix of holes 538 is larger than that of the helix of holes 438 in FIG. 11. In this way, a shaft of a guide sheath can include one or more helices of holes with varying pitch and a varying number of revolutions around the shaft.

In some examples, the holes 438 or 538 can be arranged in a double-helix pattern, such there are two helical lines of spaced apart holes 438 or 538 extending around the distal end portion of the shaft 204.

The helical pattern of holes 438 or 538 enables at least a portion of the holes 438 or 538 to be pointed “up” (anteriorly in the patient's vasculature) regardless of the rotation of the shaft 404 or 504, when inside a patient. Thus, if there is any fluid (such as air) in the shaft 404 or 504, it can always be expelled due to its buoyancy. Additionally, the helical pattern of holes 438 or 538 can ensure that there is no situation in which all or most of the holes are occluded or blocked by the patient's vasculature. For example, the curvature that the shaft 404 or 504 (or guide sheath 200) takes on within the patient's vasculature, due to the patient's vasculature anatomy, may cause the shaft 404 or 504 to be pressed against a vessel wall. The continuous revolution and rotation of the holes 438 or 538 of the helix pattern allows for at least a portion of the holes 438 or 538 to remain unblocked by the vessel wall.

The holes 238 (and any of the other holes 338, 438, and/or 538 described herein) can be configured to allow fluid pressure inside the shaft 204 to equalize. In some instances, the holes 238 can allow fluid (e.g., blood or air) to flow out of the main lumen 222 and into the surrounding environment, exterior to the shaft 204.

In some instances, each hole 238 (and any of the other holes 338, 438, and/or 538 described herein) can have a width or diameter that is specified such that air can pass therethrough from the main lumen 222 to the exterior of the shaft 204. A number of the holes 238 in the shaft 204 can also be specified such that fluid can flow out of the holes 238 in a specified location inside a patient and at various circumferential positions around the shaft 204 (e.g., around the entire circumference of the shaft 204).

In some instances, the width or diameter of the holes 238 (and any of the other holes 338, 438, and/or 538 described herein) can be in a range of 0.5-2 mm.

The holes 238 (and any of the other holes 338, 438, and/or 538 described herein) can be created in the shaft 204 in various ways. As one examples, the holes 238 can be laser cut into the formed shaft 204. Although it is not shown in FIG. 8, in some examples the shaft 204 can comprise a metal braid within a polymeric material (e.g., PEBAX). In such examples, the holes 238 can be laser cut through the polymeric material and the metal braid of the shaft 204.

In some examples, the shaft 204 can comprise a liner forming its inner surface 232. In some instances, the liner can comprise PTFE. The holes 238 can be formed in the shaft 204 such that the liner remains flush against the inner surface 232 of the shaft 204 (e.g., does not protrude radially outward), even in the regions adjacent to the holes 238.

In some examples, the holes 238 can be formed (e.g., laser cut) with a chamfer (e.g., 360-degree chamfer) on the inner surface 232 of the shaft 204. For example, as illustrated in FIG. 8 by dashed lines, the holes 238 can be formed with an angled chamfer 242 or a rounded chamfer 244 on the inner surface 232 of the shaft 204. Though only one exemplary chamfer is shown in FIG. 8, it should be noted that all the holes 238 formed in the shaft 204 could include the angled chamfer 242 or the rounded chamfer 244 on the inner surface 232.

In some instances, as shown in FIG. 8, the holes 238 can be formed (e.g., laser cut) such that they are flush with the inner surface 232 of the shaft 204.

As a result of forming the holes 238 such that they are flush or have a chamfer on the inner surface 232, no portions of the inner surface 232 of the shaft 204 protrude outward, thereby preventing from any part of a delivery apparatus (such as the prosthetic valve 150) from becoming caught on or near the holes 238 and maintaining an integrity of the liner of the shaft 204.

As introduced above, as the delivery apparatus 100 is pushed distally through the main lumen 222 of the guide catheter 200, a negative pressure (or vacuum) can be created within the main lumen 222, proximal to the implant (prosthetic heart valve 150). Thus, when the prosthetic heart valve 150 (or other implant) passes beyond the holes 238 inside the shaft 204 (e.g., toward the distal end 208 or tip of the shaft 204), vacuum pressure can be equalized by the holes 238.

In some examples, any air traveling behind the prosthetic heart valve 150 (due to being pulled along with the vacuum) can escape through the holes 238.

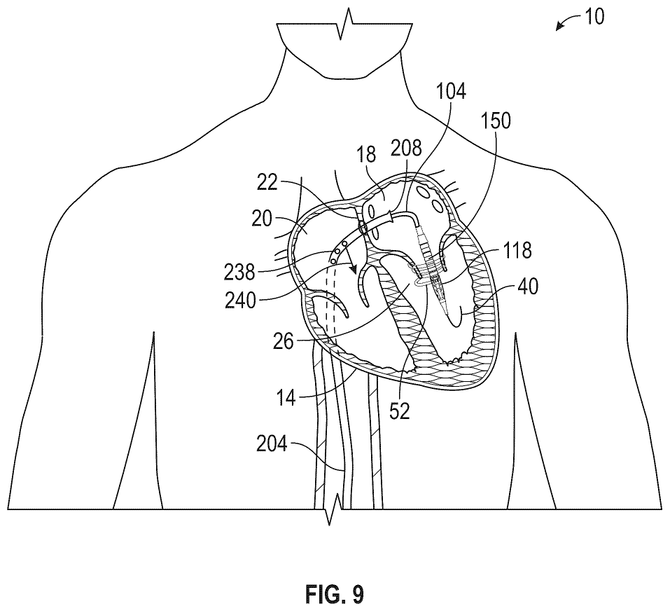

Thus, in some examples, the one or more holes 238 can be offset from the distal end 208 and further positioned in the distal end portion of the shaft 204 such that the holes 238 are disposed inside the right atrium 20 of the heart 14 when the distal end 208 of the guide sheath 200 is positioned in the left atrium 18, as shown in the schematic of FIG. 9. As a result, any air within the shaft 204 can be released into the right atrium 20 via the holes 238 as the prosthetic heart valve 150 passes by the holes 238, as shown by the arrow 240 in FIG. 9. The released air can travel from the right atrium to the lungs, for example.

In some examples, the one or more holes 238 (e.g., the most distal hole 238 of the one or more holes 238) can be axially spaced away from the distal end 208 of the guide sheath by 5-15 cm, 10-15 cm, or 5-6 cm.

In some examples, the one or more holes 238 (e.g., the most distal hole 238 of the one or more holes 238) can be axially spaced away from the distal end 208 of the guide sheath by 5-25 cm depending on a length of the shaft 204 and the target positioning of the distal end 208 inside the heart. For example, the distance between the most distal hole 238 and the distal end 208 of the guide sheath can be selected such that the most distal hole 238 is disposed within the right atrium 20.

In some examples, all of the holes 238 in the guide sheath can be positioned in the shaft 204 such that they are disposed inside the right atrium 20 when the distal end 208 of the guide sheath 200 is positioned in the left atrium 18, as shown in the schematic of FIG. 9.

In some examples, the holes 238 can be positioned along a length of the shaft 204 such that one or more holes 238 are disposed in the right atrium 20 when the distal end 208 of the guide sheath 200 is positioned in the left atrium 18, and one or more holes 238 are disposed proximal to the holes 238 in the right atrium (e.g., along another portion of the patient's vasculature). However, the placement of the holes 238 in the shaft 204 can be selected such that there is a gap (clearance) between an outer surface of the shaft 204 (at a location of each hole 238) and an inner surface of the vessel in which the guide sheath is positioned (so that air can escape out of the shaft 204 into the vessel via the holes 238).

In this way, vacuum pressure created within the shaft 204 of the guide sheath 200 during advancing the delivery apparatus through the shaft 204 can be equalized, and, in the event that there is any air traveling behind the implant on the delivery apparatus, such air can be released by the holes 238, before the distal end 208 of the shaft 204. As a result, if any air is present in the system, it can be advantageously released at an optimal location inside the patient (e.g., the right heart such that it would travel to the lungs).

Additionally, the holes in the distal end portion of the shaft can simplify priming and/or flushing processes for the guide sheath (e.g., prior to insertion of the delivery apparatus and/or during early insertion of the delivery apparatus into the proximal end of the handle 202, such as insertion into the seal housing assembly). As a result, any fluid or air present in the shaft 204, distal to the delivery apparatus, can also be expelled from the guide sheath via the holes. As a result, the guide sheath can be easier to use and the complexity of the priming or flushing process can be reduced.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a handle; and a shaft extending distally from the handle and having a main lumen, wherein a distal end portion of the shaft comprises one or more holes that extend through a wall of the shaft, between the main lumen and an exterior of the shaft, and wherein the one or more holes are spaced axially away from a distal end of the shaft.

Example 2. The delivery apparatus of any example herein, particularly example 1, wherein the one or more holes are spaced axially away from the distal end of the shaft by 5-15 cm.

Example 3. The delivery apparatus of any example herein, particularly example 1, wherein the one or more holes are spaced axially away from the distal end of the shaft by 5-25 cm.

Example 4. The delivery apparatus of any example herein, particularly any one of examples 1-3, wherein the one or more holes include a plurality of holes spaced apart from each other.

Example 5. The delivery apparatus of any example herein, particularly example 4, wherein at least a portion of holes of the plurality of holes are spaced at various circumferential locations around a circumference of the shaft.

Example 6. The delivery apparatus of any example herein, particularly either example 4 or example 5, wherein each hole of the plurality of holes is spaced axially apart from at least one other hole of the plurality of holes.

Example 7. The delivery apparatus of any example herein, particularly any one of examples 1-6, wherein each hole of the one or more holes has a length that is at least a thickness of the wall.

Example 8. The delivery apparatus of any example herein, particularly any one of examples 1-7, wherein the one or more holes are radially extending, relative to a central longitudinal axis of the shaft.

Example 9. The delivery apparatus of any example herein, particularly any one of examples 1-8, wherein the handle includes a plurality of fluid seals configured to prevent fluid flow past the plurality of fluid seals.

Example 10. The delivery apparatus of any example herein, particularly example 9, wherein the handle includes a flush lumen that extends between the main lumen and a flush port coupled to a housing of the handle, and wherein the flush lumen is disposed distal to the plurality of fluid seals.

Example 11. The delivery apparatus of any example herein, particularly either example 9 or example 10, wherein the shaft extends within the handle, to the plurality of fluid seals.

Example 12. The delivery apparatus of any example herein, particularly any one of examples 1-11, wherein the handle includes a main body portion, and wherein the main body portion contains flex mechanisms that are configured to adjust a curvature of the distal end portion of the shaft.

Example 13. The delivery apparatus of any example herein, particularly example 12, wherein the handle further includes a rotatable knob operatively coupled to the flex mechanisms.

Example 14. A delivery assembly comprising: an implant catheter; and a guide catheter comprising: a handle; and a shaft extending distally from within the handle and having a main lumen that is configured to receive a portion of the implant catheter therethrough, wherein a distal end portion of the shaft includes a plurality of through-holes therein that each extend through a wall of the shaft, between the main lumen and an outer surface of the shaft, and wherein the plurality of through-holes is spaced axially away from a distal end of the shaft.

Example 15. The delivery assembly of any example herein, particularly example 14, wherein the through-holes of the plurality of through-holes are spaced axially away from the distal end of the shaft by 5-15 cm.

Example 16. The delivery assembly of any example herein, particularly either example 14 or example 15, wherein the through-holes of the plurality of through-holes are spaced apart from each other along a portion of the shaft.

Example 17. The delivery assembly of any example herein, particularly example 16, wherein the through-holes of the plurality of through-holes are spaced circumferentially apart at various circumferential positions around the shaft.

Example 18. The delivery assembly of any example herein, particularly either example 16 or example 17, wherein each through-hole of the plurality of through-holes is spaced axially apart from another, adjacent through-hole of the plurality of through-holes.

Example 19. The delivery assembly of any example herein, particularly any one of examples 14-18, wherein the handle includes a plurality of fluid seals configured to prevent fluid flow past the plurality of fluid seals, and wherein the handle includes a flush lumen that extends between the main lumen and a flush port coupled to a housing of the handle, the flush lumen disposed distal to the plurality of fluid seals.

Example 20. The delivery assembly of any example herein, particularly any one of examples 14-19, wherein each through-hole of the plurality of through-holes is formed flush with an inner surface of the shaft.

Example 21. The delivery assembly of any example herein, particularly any one of examples 14-20, wherein the shaft is a steerable shaft, and wherein the handle comprises a flex mechanism configured to adjust a curvature of the distal end portion of the shaft.

Example 22. The delivery assembly of any example herein, particularly any one of examples 14-21, wherein the implant catheter is configured to deliver a prosthetic heart valve mounted around the distal end portion of the implant catheter.

Example 23. A guide sheath comprising: a handle comprising a seal housing assembly including one or more fluid seals; and a shaft extending within and distally from the handle and having a main lumen that extends within the housing and through the seal housing assembly, wherein a distal end portion of the shaft includes a plurality of holes that extend through a thickness of a wall of the shaft, between the main lumen and an outer surface of the shaft, and wherein the plurality of holes is spaced axially away from a distal end of the shaft.

Example 24. The guide sheath of any example herein, particularly example 23, wherein the plurality of holes is spaced axially away from the distal end of the shaft by 5-25 cm.

Example 25. The guide sheath of any example herein, particularly either example 23 or example 24, wherein at least a portion of the holes of the plurality of holes are spaced circumferentially apart at various circumferential positions around the shaft.

Example 26. The guide sheath of any example herein, particularly any one of examples 23-25, wherein each hole of the plurality of holes is spaced axially apart from at least one adjacent hole of the plurality of holes.

Example 27. The guide sheath of any example herein, particularly any one of examples 23-26, wherein at least a portion of the plurality of holes are radially extending, relative to a central longitudinal axis of the shaft.

Example 28. The guide sheath of any example herein, particularly any one of examples 23-27, wherein the handle includes a flush lumen that extends between the main lumen and a flush port coupled to a housing of the handle, and wherein the flush lumen is disposed distal to the seal housing assembly.

Example 29. The guide sheath of any example herein, particularly any one of examples 23-28, wherein the handle includes a main body portion, and wherein the main body portion contains flex mechanisms that are configured to adjust a curvature of the distal end portion of the shaft.

Example 30. A method comprising: inserting a shaft of a guide catheter into a vessel of a patient and advancing a distal end portion of a shaft of the guide shaft into the heart of the patient such that a distal end of the shaft is positioned in the left atrium of the heart and one or more through-holes in the distal end portion of the shaft are positioned in the right atrium of the heart; inserting a distal end portion of a first implant catheter into a proximal end of the guide catheter and pushing the distal end portion of the first implant catheter through a main lumen of the guide catheter toward a target implantation site for a prosthetic medical device mounted on the distal end portion of the first implant catheter; and as the prosthetic medical device mounted on the distal end portion of the first implant catheter passes by the one or more through-holes in the shaft, releasing fluid traveling behind the prosthetic medical device into the right atrium through the one or more through-holes.

Example 31. The method of any example herein, particularly example 30, wherein the fluid is air.

Example 32. A method comprising sterilizing the prosthetic heart valve, apparatus, guide sheath, and/or assembly of any example.

Example 33. The delivery apparatus of any example herein, particularly any one of examples 4-6, wherein the plurality of holes is arranged in a helical pattern around the shaft, along a portion of a length of the shaft.

Example 34. The delivery apparatus of any example herein, particularly example 33, wherein the helical pattern of the plurality of holes forms at least two revolutions around the shaft.

Example 35. The delivery apparatus of any example herein, particularly any one of examples 4-6 and 33-34, wherein at least one hole of the plurality of holes has a different diameter than another hole of the plurality of holes.

Example 36. The delivery assembly of any example herein, particularly any one of examples 14-22, wherein the plurality of through-holes is arranged in a helical pattern around the shaft, along a portion of a length of the shaft, such that each through-hole is spaced axially and circumferentially apart from at least one adjacent through-hole.

Example 37. The delivery assembly of any example herein, particularly example 36, wherein the helical pattern of the plurality of holes forms at least two revolutions around the shaft.

Example 38. The guide sheath of an example herein, particularly any one of examples 23-29, wherein the plurality of holes is arranged in a helical pattern around the shaft, along a portion of a length of the shaft.

Example 39. The guide sheath of any example herein, particularly example 38, wherein the helical pattern of the plurality of holes forms at least two revolutions around the shaft.

The features described herein with regard to any example can be combined with other features described in any one or more of the other examples, unless otherwise stated. For example, any one or more of the features of one guide catheter can be combined with any one or more features of another guide catheter. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus comprising:
a handle; and
a shaft extending distally from the handle and having a main lumen, wherein a distal end portion of the shaft comprises one or more holes that extend through a wall of the shaft, between the main lumen and an exterior of the shaft, and wherein the one or more holes are spaced axially away from a distal end of the shaft.

2. The delivery apparatus of claim 1, wherein the one or more holes are spaced axially away from the distal end of the shaft by 5-25 cm.

3. The delivery apparatus of claim 1, wherein the one or more holes include a plurality of holes spaced apart from each other.

4. The delivery apparatus of claim 3, wherein at least a portion of holes of the plurality of holes are spaced at various circumferential locations around a circumference of the shaft.

5. The delivery apparatus of claim 3, wherein each hole of the plurality of holes is spaced axially apart from at least one other hole of the plurality of holes.

6. The delivery apparatus of claim 3, wherein the plurality of holes is arranged in a helical pattern around the shaft, along a portion of a length of the shaft.

7. The delivery apparatus of claim 1, wherein the handle includes a plurality of fluid seals configured to prevent fluid flow past the plurality of fluid seals.

8. The delivery apparatus of claim 7, wherein the handle includes a flush lumen that extends between the main lumen and a flush port coupled to a housing of the handle, and wherein the flush lumen is disposed distal to the plurality of fluid seals.

9. The delivery apparatus of claim 7, wherein the shaft extends within the handle, to the plurality of fluid seals.

10. A delivery assembly comprising:
an implant catheter; and
a guide catheter comprising:
a handle; and
a shaft extending distally from within the handle and having a main lumen that is configured to receive a portion of the implant catheter therethrough, wherein a distal end portion of the shaft includes a plurality of through-holes therein that each extend through a wall of the shaft, between the main lumen and an outer surface of the shaft, and wherein the plurality of through-holes is spaced axially away from a distal end of the shaft.

11. The delivery assembly of claim 10, wherein the through-holes of the plurality of through-holes are spaced axially away from the distal end of the shaft by 5-15 cm.

12. The delivery assembly of claim 10, wherein the through-holes of the plurality of through-holes are spaced circumferentially apart at various circumferential positions around the shaft.

13. The delivery assembly of claim 10, wherein each through-hole of the plurality of through-holes is spaced axially apart from another, adjacent through-hole of the plurality of through-holes.

14. The delivery assembly of claim 10, wherein the plurality of through-holes is arranged in a helical pattern around the shaft, along a portion of a length of the shaft, such that each through-hole is spaced axially and circumferentially apart from at least one adjacent through-hole.

15. The delivery assembly of claim 10, wherein the shaft is a steerable shaft, and wherein the handle comprises a flex mechanism configured to adjust a curvature of the distal end portion of the shaft.

16. The delivery assembly of claim 10, wherein the implant catheter is configured to deliver a prosthetic heart valve mounted around the distal end portion of the implant catheter.

17. A guide sheath comprising:
a handle comprising a seal housing assembly including one or more fluid seals; and
a shaft extending within and distally from the handle and having a main lumen that extends within the housing and through the seal housing assembly, wherein a distal end portion of the shaft includes a plurality of holes that extend through a thickness of a wall of the shaft, between the main lumen and an outer surface of the shaft, and wherein the plurality of holes is spaced axially away from a distal end of the shaft.

18. The guide sheath of claim 17, wherein the plurality of holes is spaced axially away from the distal end of the shaft by 5-25 cm.

19. The guide sheath of claim 17, wherein the plurality of holes is arranged in a helical pattern around the shaft, along a portion of a length of the shaft.

20. The guide sheath of claim 19, wherein the helical pattern of the plurality of holes forms at least two revolutions around the shaft.
